(19) United States
(12) Patent Application Publication
(10) Pub. No.: US 2008/0124778 A1
(43) Pub. Date: May 29, 2008

(54) Electroporation Cuvette with High Field Intensity and Field-Induced Agitation

(75) Inventor: Charles W. Ragsdale, Concord, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BIO-RAD LABORATORIES, INC., Hercules, CA (US)

(21) Appl. No.: 11/564,738

(22) Filed: Nov. 29, 2006

(51) Int. Cl. C12N 13/00; C12M 1/42

(52) U.S. Cl. 435/173.6; 435/285.2

(57) ABSTRACT

An electroporation cuvette is constructed as a cylinder with a filament electrode at the cylinder axis and one or more peripheral electrodes on the internal cylinder wall. An additional cuvette design is one with a plurality of peripheral electrodes distributed at various locations around the circumference of the cuvette wall and no central electrode. The various arrangements of electrodes lead to electric field vectors that are non-uniform and of varying orientation, producing high field intensities and agitation of the cell suspension in the cuvette during electroporation.

### BACKGROUND OF THE INVENTION

1. Field of the Invention

This invention resides in the field of electroporation, a process for inserting exogenous molecular species into membranous structures such as biological cells, liposomes, or vesicles, by suspending the structures is a liquid solution of the exogenous species and applying an electric field to the suspension.

2. Description of the Prior Art

Electroporation is a valuable technique in the performance of a wide variety of investigations and procedures performed by research biologists and biochemists. These include siRNA experiments and research using cDNA libraries, as well as clinical procedures and any other procedures involving the transfection of cells, liposomes, or vesicles with exogenous molecules. The process is susceptible to variability in its effectiveness, however, and because of this susceptibility, together with the need to avoid or minimize damage to the membranous structures, the efficiency of the process is typically very low. One factor that affects the efficiency is the distribution of the structures within the suspension and the tendency of certain structures to aggregate into clumps. Another factor is the uniformity or lack of uniformity of the electric field, and a third is the tendency of individual structures to shield other structures from the electric field and thereby limit the exposure of the shielded structures to the field and to the exogenous species.

When electroporation is performed in individual cuvettes, the typical cuvette is either of square or rectangular cross section, and the electrodes are plates on opposite sides of the cuvette. The efficiency of the procedure in terms of the number of membranous structures successfully transfected vs. the amount of electrical energy consumed is limited by spatial considerations, notably the volume between the electrodes and restrictions on the movement of the membranous structures between the electrodes, and by the lack of uniformity of the electric field throughout the cuvette. The present invention seeks to address these limitations.

### SUMMARY OF THE INVENTION

The present invention resides in certain cuvette constructions that variously provide a high proportion of suspension volume to electric energy consumption, the ability to vary the direction of the electric field vector during a single pulse or in successive pulses and thereby approach individual membranous structures from different angles, and the ability to impose directional forces on the suspended membranous structures to cause agitation, rotation, reorientation, and redistribution of the structures. In one set of embodiments, the invention resides in a cuvette in the form of a liquid-retaining receptacle having a base and a cylindrical wall that is symmetrical about a longitudinal axis, where the electrodes consist of one or more periphery electrodes along the cylindrical wall and a filament electrode in the center along the longitudinal axis. In some of the embodiments in this set, a single periphery electrode extends around the full circumference of the receptacle wall, establishing an electric field in the annular space the periphery electrode and the filament electrode. This construction provides a relatively short distance between opposing electrodes (periphery and filament) and therefore a potentially higher field density than that of a cuvette with an equal cross sectional area and the same charges on the electrodes but with plate electrodes on opposing walls. In other embodiments within this first set, the single periphery electrode is replaced by a series of plate or strip periphery electrodes distributed along the circumference of the receptacle wall, each electrode of the series individually connected to a power source for independent energization. In this arrangement, an electric field can be established between the filament electrode and any individual electrode or a subgroup of electrodes, in succession, moving the location of the electric field around the axis of the cuvette and rotating the electric field vector. In another set of embodiments, the periphery electrodes are divided into upper and lower electrodes spaced apart along the cuvette axis, individually connected to a power supply to allow the electrodes to apply a force with an axial component to the membranous structures. The periphery electrodes in this set are either a single upper and a single lower electrode or a series of upper electrodes and a series of lower electrodes. The series embodiments allows one to select among several different pairs of electrodes for energization to produce electric field vectors in a plurality of directions. A still further set of embodiments are those in which the filament electrode is connected to a source of mechanical vibration and thereby adds mechanical agitation to the forces imposed on the suspended membranous structures.

A still further set of embodiments are those in which the filament electrode is eliminated entirely and a series of peripheral electrodes are distributed around the circumference of the cuvette wall, the peripheral electrodes individually connected to a power source for independent energization, allowing a multitude of pairings of energized electrodes, each producing its own distinct electric field vector.

The structural features and operation of these and other embodiments of the invention will be better understood by the description that follows.

### BRIEF DESCRIPTION OF THE DRAWINGS

FIG. 1 is a perspective view of an electroporation cuvette in accordance with the present invention, in the form of a circular cylinder with an axial filament electrode.

FIG. 2 is a perspective view of a second electroporation cuvette in accordance with the present invention, in the form of a cylinder of hexagonal cross section, again with an axial filament electrode.

FIG. 3 is a perspective view of a third electroporation cuvette in accordance with the present invention, representing a variation of the cuvette of FIG. 2.

FIG. 4 is a perspective view of a fourth electroporation cuvette in accordance with the present invention, without an axial filament electrode.

### DETAILED DESCRIPTION OF THE INVENTION AND PREFERRED EMBODIMENTS

The terms “cylindrical” and “cylinder,” as used herein in reference to the wall of the receptacle that serves as the cuvette, denote the surfaces traced by a straight line moving parallel to the longitudinal axis of the receptacle and intersecting a fixed planar closed outline. The closed outline can be a polygon with straight sides, a curvilinear figure such as a circle or ellipse, or a figure with a combination of straight segments and curvilinear segments. The cylinder can be symmetrical about the longitudinal axis or asymmetrical, although cylinders that are symmetrical, either about the axis or about a plane containing the axis, are preferred. For cylinders with curvilinear contours, circular cylinders (i.e., cylinders with circular cross section) are preferred. For cylinders with polygonal cross sections, preferred polygons are those whose sides are all of equal dimensions and are all equidistant from the longitudinal axis. Polygons of five to twelve sides are preferred in certain embodiments, while polygons of six sides or more, and particularly six to eight sides, are preferred in certain other embodiments. Cuvettes of hexagonal cross section are the most preferred among those of polygonal shape.

The term “filament electrode” denotes a small-diameter rod or wire that has sufficient stiffness to stand upright when secured only at its lower end. In preferred embodiments, particularly those in which the filament is connected to a mechanical vibration source, the filament electrode is also resilient, i.e., responsive to a vibratory force by bowing but returning to a fully extended configuration when at rest. The filament electrode, when present, is at least approximately aligned with the axis of the cylinder and can be secured to the receptacle at either end or both ends. Preferably, the filament is secured to the base of the receptacle only, extending into the receptacle interior to terminate in an unsecured tip.

While the features defining this invention in each of its various embodiments are capable of implementation in a variety of constructions, the invention as a whole will be best understood by a detailed examination of specific embodiments. The drawings hereto depict such embodiments and are discussed below.

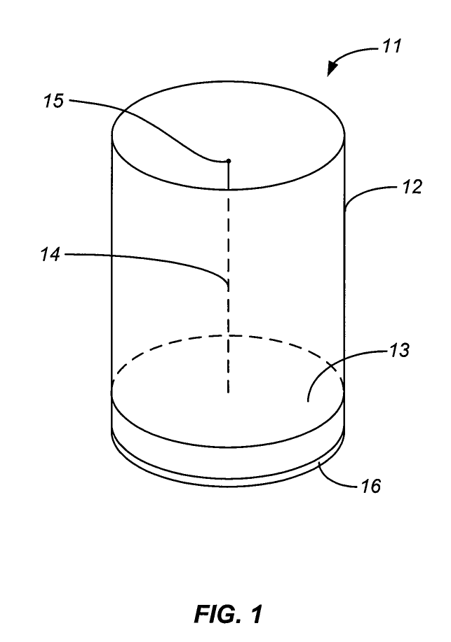

FIG. 1 depicts a cuvette 11 in the form of a circular cylinder. As pictured in the Figure, the cuvette 11 is an open-top receptacle with a cylindrical wall 12 that has a circular cross section, and a base 13. The inner surface of the entire cylindrical wall 12 is plated with a conductive material to serve as a periphery electrode. Along the central axis of the cylinder (i.e., the longitudinal axis of the cuvette) is a filament electrode 14. The electric field that results when the electrodes are charged with opposite polarities fills the entire volume of the cuvette around the filament electrode 14 and is radially directed. Since the distance between the filament electrode and the periphery electrode is only half the lateral dimension (i.e., the diameter) of the cuvette, the field intensity is twice the intensity that would be achieved with a rectangular cuvette of the same lateral dimension, and approximately twice the intensity that would be achieved with a rectangular cuvette of the same internal volume. The smaller distance between electrodes also reduces the degree of possible shielding of one cell by other cells.

In addition, the radial shape of the electric field vector produces fan-shaped field lines through individual cells suspended in the cuvette. This nonuniformity of field produces an electric field gradient and in some cases an electric moment sufficient to cause movement of the cells. This movement can reduce cell aggregation, continuously vary the orientations of the cells to expose different portions of the membrane of a given cell to the electric field, and produce a changing electric field environment immediately surrounding the cell.

The base 13 of the cuvette in this embodiment is an ultrasonic crystal that imparts ultrasonic vibrations to the filament electrode 14. The filament electrode responds freely to the vibrations since the upper end 15 of the electrode is unsecured or floating. The ultrasonic crystal 13 may be of the type used in ultrasonic nebulizers or in medical implants for the monitoring of ventricular function or for various clinical and physiological studies. Ultrasonic crystals are known in the art of medical devices, and can be driven by any of various types of transducers, including piezoelectric and magnetic transducers. Other means of creating vibrations and transmitting them to the filament electrode are: fabricating the filament from magnetic material and applying a varying current to a coil surrounding an exposed end of the filament or surrounding the outside of the cuvette; attaching a mechanical actuator to the filament through a mechanical linkage, the actuator being a motor or piston driven by an air or fluid drive; and embedding a miniature motor at the base of the filament, the motor driven by energy derived from detected RF fields impinging on the cuvette.

In the cuvette shown in FIG. 1, bottom surface of the ultrasonic crystal 13 is coated or plated with an electrical conductor 16 serving as a lead for the filament electrode 14, which extends through the crystal to the conductor. The periphery electrode 12 is connected to a separate lead.

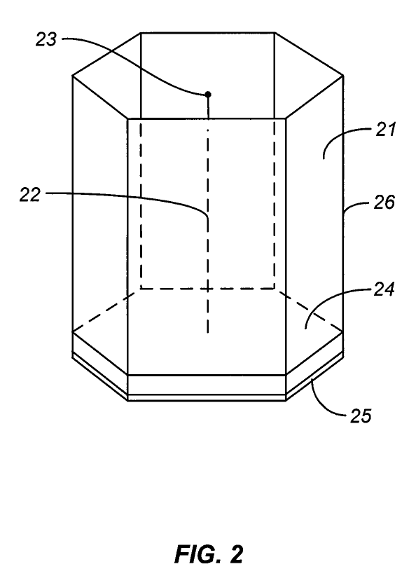

The cuvette of FIG. 2 differs from that of FIG. 1 by having a cylindrical wall 21 in which the cylinder cross section is a polygon, in this case a hexagon. A filament electrode 22 extends upward along the longitudinal axis of the cuvette, terminating at a floating tip 23 within the interior of the cuvette. At the base of the cuvette is an ultrasonic crystal 24 whose lower surface is plated with a conductor 25 to which the filament electrode is connected. The entire inner surface of the hexagonal cylinder 21 can be plated with a conductor to form a single peripheral electrode fully surrounding the cuvette interior. Alternatively, plating can be done on every second side of the hexagonal cylinder for a total of three sides with peripheral electrodes alternating with three sides bearing no electrodes. In a further alternative, each of the six sides of the cylinder can be plated, the plated areas separated by gaps along the lines 26 adjoining each pair of adjacent sides, with a total of six periphery electrodes rather than one. In either case, each periphery electrode can then be connected to a separate electric lead, allowing the electrodes to be operated independently. Electric field vectors of different orientations can then be applied in sequence, adding to the mobility of the suspended cells in addition to varying the direction of exposure.

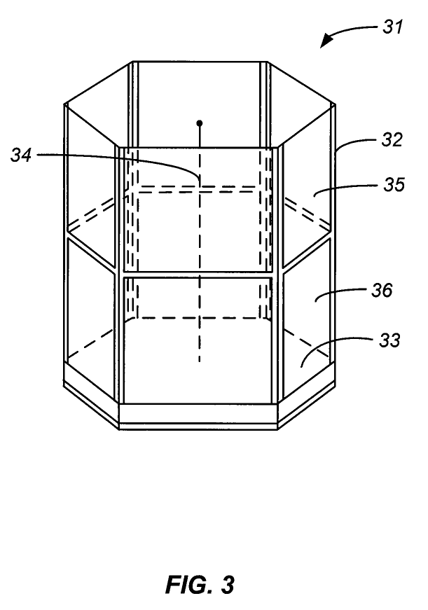

The cuvette of FIG. 3 adds a further dimension to the electric field vectors and the directions of movement of the cells. The cuvette 31 has a peripheral wall 32 of hexagonal cross section similar to that of FIG. 2, and other features of FIG. 2, including a hexagonal ultrasonic crystal 33 at its base and a filament electrode 34 along its longitudinal axis. Periphery electrodes are present on all six sides of the cuvette, but are also divided into upper 35 and lower 36 electrodes, for a total of twelve periphery electrodes. By energizing the upper electrodes without the lower electrodes or energizing the upper and lower electrodes at different times or in various combinations, electric fields are readily created with vectors that have axial components as well as radial components, adding further moments to the suspended cells and increasing the possibilities for agitation.

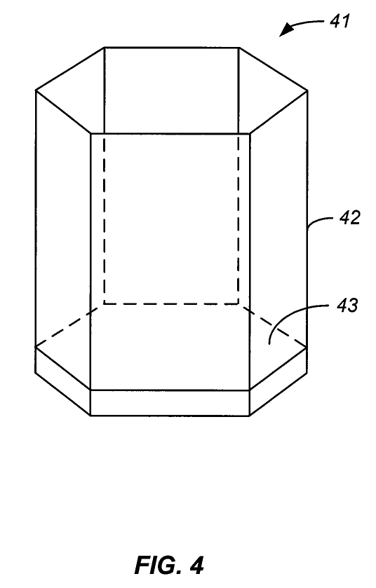

The cuvette 41 of FIG. 4 is identical to that of FIG. 2 except for the elimination of the filament electrode. Here as well, the cuvette has a side wall 42 of hexagonal cross section and a base 43. The only electrodes are the periphery electrodes, which are all affixed to the sides of the cuvette in a manner allowing adjacent electrodes to be electrically charged independently of each other. This can be achieved by placing the electrodes on alternating sides or on adjacent sides with gaps along the lines where adjacent sides meet. Electrical connections joining the electrodes to power sources can be arranged to energize electrodes on opposing sides, and the electrodes can be energized sequentially to cause rotation and agitation of the suspended cells.

In use, the cuvettes of the present invention are charged with a suspension of membranous structures in a solution of the exogenous species, and electroporation is conducted under conditions and procedures known in the art. Descriptions of the procedure and operating conditions are found in literature supplied by Bio-Rad Laboratories, Inc. (Hercules, Calif., USA), entitled Gene Pulser Xcell System, Bulletin No. 2750US/EG Rev. B, published in January 2004, which sets forth descriptions of apparatus, materials, and methods for electroporation of various kinds of cells, both eukaryotic and prokaryotic. Further descriptions are found in Chassy, B. M., et al., “Transformation of lactobacillus casei by electroporation,” FEMS Microbiol. Lett. 44:173-177 (1987) and Powell, I. B., et al., “A Simple and Rapid Method for Genetic Transformation of Lactic Streptococci by Electroporation,” Appl. Environ. Microbiol. 54(3): 655-660 (March 1988); Potter, H., et al., “Enhancer-dependent expression of human kappa immunoglobulin genes introduced into mouse pre-B lymphocytes by electroporation,” Proc. Natl. Acad. Sci. USA 81: 7161-7165 (1984); Shivarova, N., et al., “Microbiological implications of electric field effects. VII. Stimulation of plasmid transformation of protoplasts by electric field pulses,” Zeitschrift Allge. Mikro. 23:595-599 (1983); and Miller, J. F., et al., “High voltage electroporation of bacteria: genetic transformation of Campylobacier jejuni with plasmid DNA,” Proc. Natl. Acad. Sci. USA 85: 856-860 (1988). Patents that disclose electroporation cells, materials, and methods include Dower, W. J., U.S. Pat. No. 4,910,140, Mar. 20, 1990, and U.S. Pat. No. 5,186,800, Feb. 16, 1993, Korenstein, R., et al., U.S. Pat. No. 5,964,726, Oct. 12, 1999; Thompson, J. R., U.S. Pat. No. 5,879,891, Mar. 9, 1999; and Greener, A. L., et al., U.S. Pat. No. 6,586,249, Jul. 1, 2003.

Cells that are intended for transformation by electroporation are known as electrocompetent cells, electrocompetency being achieved by suspending normal cells in a low-conductivity medium to prevent arcing during electroporation. Electrocompetent cells are available commercially and are typically sold in microtubes. To perform electroporation, the user first transfers the cell suspension from the microtube to an empty tube, then adds the nucleic acid or other exogenous species, mixes the suspension to distribute the specie, and transfers the combined suspension to a cuvette for electroporation. Alternatively, the transformation agent is added to the electrocompetent cell suspension in the microtube, and the combined suspension is then placed in the cuvette.

While the foregoing description describes various alternatives to the components shown in the Figures, still further alternatives will be apparent to those skilled in the art and are within the scope of the invention.

In the claims below, the terms “a” and “an” are intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. An electroporation cuvette comprising:
a liquid-retaining receptacle comprising a longitudinal axis, a cylindrical wall symmetrically arranged about said longitudinal axis, and a base,
a periphery electrode affixed to said cylindrical wall, and
a filament electrode affixed to said receptacle and extending along said longitudinal axis,
said periphery and filament electrodes positioned such that, when said receptacle is charged with liquid and said periphery and filament electrodes are electrically charged with opposing polarities, an electrical potential is established between said periphery and filament electrodes and through said liquid.

2. The electroporation cuvette of claim 1 wherein said filament electrode is affixed to said base and terminates in an unsecured tip within said receptacle.

3. The electroporation cuvette of claim 1 wherein said cylindrical wall is a circular cylinder, and said periphery electrode spans the full circumference of said circular cylinder.

4. The electroporation cuvette of claim 1 wherein said cylindrical wall has a polygonal cross section, all sides of which are of equal dimensions and equidistant from said longitudinal axis.

5. The electroporation cuvette of claim 4 wherein said polygonal cross section is a polygon of five to twelve sides.

6. The electroporation cuvette of claim 5 wherein said polygonal cross section is a polygon of six to eight sides.

7. The electroporation cuvette of claim 5 wherein said cylindrical wall has a hexagonal cross section.

8. The electroporation cuvette of claim 4 wherein said polygonal cross section is a polygon having an even number of sides of at least six in number, said electroporation cuvette comprising a plurality of periphery electrodes, one such periphery electrode on each of alternating sides of said polygon.

9. The electroporation cuvette of claim 4 comprising a plurality of periphery electrodes, one such periphery electrode on each side of said polygon, said electroporation cuvette further comprising means for energizing said periphery electrodes independently.

10. The electroporation cuvette of claim 4 comprising a plurality of periphery electrodes divided into first and second sets, with a pair of periphery electrodes, one from each set, axially positioned on each side of said polygon, said electroporation cuvette further comprising axial potential means for energizing said first set of periphery electrodes independently of said second set of periphery electrodes.

11. The electroporation cuvette of claim 1 further comprising electrode vibrating means for causing vibratory movement of said filament electrode.

12. The electroporation cuvette of claim 11 wherein said electrode vibrating means is an ultrasonic transducer.

13. The electroporation cuvette of claim 11 wherein said filament electrode is affixed to said base and terminates in an unsecured tip within said receptacle, and said electrode vibrating means is an ultrasonic crystal joined to filament electrode at said base.

14. An electroporation cuvette comprising:
a liquid-retaining receptacle comprising a cylindrical wall and a base, said cylindrical wall having a polygonal cross section of at least six sides,
electrodes affixed to said sides of said cylindrical wall in a manner allowing adjacent electrodes to be electrically charged independently of each other, and
means for energizing electrodes on opposing sides of said cylindrical wall at opposite polarities.

15. The electroporation cuvette of claim 14 wherein said electrodes consist of first and second sets of electrodes affixed to said sides of said cylindrical wall with one electrode per side and arranged such that electrodes of said first set alternate with electrodes of said second set, and energizing means for energizing said electrodes of said first and second sets such that electrodes of said first set are at opposite polarities of said electrodes of said second set.

16. The electroporation cuvette of claim 15 wherein said energizing means comprises means for energizing electrodes of said first set in succession and means for energizing electrodes of said second set in succession at opposite polarities to electrodes of said first set.

17. A process for transfecting a membraneous structure with a species exogenous to said structure, said process comprising (a) charging a cuvette with a suspension of said membranous structure in a liquid solution of said species, said cuvette comprising:
a liquid-retaining receptacle comprising a longitudinal axis, a cylindrical wall symmetrically arranged about said longitudinal axis, and a base,
a periphery electrode affixed to said cylindrical wall, and
a filament electrode affixed to said receptacle and extending along said longitudinal axis,
and (b) electrically charging said periphery and filament electrodes at opposing polarities to expose said suspension in said cuvette to an electric field sufficient to cause said transfection.

18. The process of claim 17 wherein said cylindrical wall is a circular cylinder, and said periphery electrode spans the full circumference of said circular cylinder.

19. The process of claim 17 wherein said cylindrical wall has a polygonal cross section, all sides of which are of equal dimensions and equidistant from said longitudinal axis.

20. The process of claim 17 wherein said polygonal cross section is a polygon having an even number of sides of at least six in number, said electroporation cuvette comprising a plurality of periphery electrodes, one such periphery electrode on each of alternating sides of said polygon.

21. A process for transfecting a membraneous structure with a species exogenous to said structure, said process comprising (a) charging a cuvette with a suspension of said membranous structure in a liquid solution of said species, said cuvette comprising:
a liquid-retaining receptacle comprising a cylindrical wall and a base, said cylindrical wall having a polygonal cross section of at least six sides,
electrodes affixed to said sides of said cylindrical wall in a manner allowing adjacent electrodes to be electrically charged independently of each other, and
(b) electrically charging electrodes on opposing sides of said cylindrical wall to expose said suspension in said cuvette to an electric field sufficient to cause said transfection.
